(19) United States
(12) Patent Application Publication
(10) Pub. No.: US 2007/0270646 A1
(43) Pub. Date: Nov. 22, 2007

(54) Cystoscope and disposable sheath system

(76) Inventor: Perry Weiner, Lafayette Hill, PA (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/438,032

(22) Filed: May 19, 2006

(51) Int. Cl. A61B 1/00; A61B 1/04

(52) U.S. Cl. 600/121

(57) ABSTRACT

A disposable sheath to be used in conjunction with a cystoscope or endoscope. The disposable sheath including an external wall having a proximal end and a distal end. The sheath also including a first channel disposed within the external wall. The first channel including an interior wall defining a hollow chamber and a longitudinal axis passing through a center of the first channel. The first channel having an enlarged integral portion or abutment disposed within the hollow chamber. The enlarged integral portion or the abutment having wall that extends from the interior wall of the first channel toward the longitudinal axis that passes through the center of the first channel to guide the cystoscopic accessory.

### BACKGROUND OF THE INVENTION

The use of cystoscopes and endoscopes for diagnostic and therapeutic indications is rapidly expanding. To improve performance, cystoscopes and endoscopes have been optimized to best accomplish their purpose. Therefore, there are upper endoscopes for examination of the esophagus, stomach, and duodenum; colonoscopes for examining the colon; angioscopes for examining blood vessels, bronchoscopes for examining the bronchi; laparoscopes for examining the peritoneal cavity; and arthroscopes for examining joint spaces. Cystoscopes are used to examine the urinary bladder, and ureteroscopes are used to examine the ureter and kidney. The discussion which follows will apply to all of these types of endoscopes.

Instruments to examine the rectum and sigmoid colon, known as flexible sigmoidoscopes, are good examples of the usefulness of endoscopic technology. These devices are expensive, and they are used in a contaminated environment for a brief procedure (5-10 minutes) to screen symptomatic and asymptomatic patients for colon and rectal cancer. Typically, these endoscopes have a flexible insertion tube that is inserted into a patient during an endoscopic procedure. During insertion, the position of the distal end of the insertion tube is controlled by control devices on the endoscope's handle at the proximal end of the insertion tube. The distal end of the insertion tube must be sufficiently flexible to bend through tight radius corner during insertion.

The endoscopes also have multiple channels that extend along the length of the endoscope and come into contact with body tissues and fluids during the procedure. These channels allow air insufflation into a body cavity, water flow to wash the endoscope's tip, suction through the tip, and biopsies to be taken. The channels must be sufficiently flexible to bend with the insertion tube through the short radius turns. The channels also must substantially maintain their cross-sectional shape and resist kinking around the tight turns to assure continuous air flow, water flow, suction, and biopsy capabilities throughout the procedure.

Those endoscopes having integral working channels along the neutral bending plane are severely contaminated during an endoscopic procedure. Proper cleaning and sterilization of the insertion tube and the integral work channels is very laborious and costly, thereby reducing the cost effectiveness of performing the therapeutic or diagnostic endoscopic procedures. Additionally, cold soaking solutions currently in use have the potential hazard or allergenicity and airway irritation to those handling or working near the solution.

Disposable endoscopic sheaths have been developed to alleviate the problem of cleaning and sterilizing the working channels. The disposable sheaths fit over the insertion tube and completely isolate an insertion tube from the contaminating environment. The isolated insertion tube may not have integral working channels, but may contain a visual imaging device, illumination devices, and the control wires that bend the insertion tube's distal end. Working channels, such as air, water, and biopsy/suction channels, are disposed within the sheath adjacent to the isolated insertion tube and are open to the sheath's distal end to allow passage of air, water, suction or endoscopic accessories through the channels.

### SUMMARY OF THE INVENTION

The present invention is directed to a disposable sheath to be used in conjunction with a cystoscope or endoscope. The disposable sheath includes an external wall having a proximal end and a distal end. The sheath may also include a first channel disposed within the external wall. The first channel includes an interior wall defining a hollow chamber and a longitudinal axis passing through a center of the first channel. The first channel has a redirecting portion at the distal end for redirecting a cystoscopic accessory in a desired direction as it moves through the redirecting portion.

The redirecting portion preferably directs the cystoscope accessory toward a working end of the cystoscope that is disposed within the sheath. In certain embodiments the redirecting portion includes a slanted wall.

In one aspect of the present invention the redirecting portion includes an abutment defining the slanted wall and a notch opposite the abutment. The notch at least partially defined by an edge of the interior wall disposed within the slanted wall, both of which cooperate therewith to guide the cystoscope accessory in the desired direction.

On one aspect of the present invention the disposable sheath further includes a second channel having a second interior wall defining a second hollow chamber and a transparent wall proximate the distal end of the external wall. The second channel is adapted to fit over a working end of a cystoscope such that at least a portion of the working end of the cystoscope is isolated from an external environment.

The first channel may include a first portion, which has a circular cross-section. The first channel may include a first section that extends in a first plane and a second section that extends in a second plane. The second plane may be angled relative to the first plane. The external wall may be comprised of a polymeric material. The redirecting portion may be integrally formed with the external wall.

In one aspect of the present invention the disposable sheath to be used in conjunction with a cystoscope or endoscope, the disposable sheath includes a first inner lumen includes a proximal end and a distal end. The first inner lumen further including a hollow chamber defined by an interior wall and an open end proximate the distal end, wherein an object may traverse from the proximal end of the hollow chamber toward the distal end in a first direction. The inner lumen including an abutment disposed adjacent the hollow member, which includes an edge. Wherein, when the object translating through the hollow chamber in a first direction contacts the edge of the abutment the object is redirected to a second direction.

The sheath may also include a second inner lumen including a proximal end, a distal end and a hollow chamber defined by an interior wall. The second inner lumen further having a closed transparent end positioned adjacent the distal end of the second inner lumen, wherein the second inner lumen is adapted to house a working end of a cystoscope such that the working end of the cystoscope is tightly surrounded and isolated from an external environment. The cross-section of the hollow chambers of both the first inner lumen and the second inner lumen may be substantially circular for at least a portion of each of the hollow chambers.

The disposable sheath may also include a cystoscope having a working end disposed therein. The working end of said cystoscope including an imaging device for viewing an image. When the working end of the cystoscope is disposed in said second inner lumen the imaging device images an image of an area beyond the distal end of the second inner lumen such that one my view the area. The object translating though the hollow chamber of the first inner lumen and redirected by the abutment to a second direction, the second direction steers the object towards the area being viewed by the imaging device.

In one method according to the present invention, the method may include the steps of placing a working end of a cystoscope into a first inner lumen of a disposable sheath. Next inserting a portion of the cystoscope disposed within the disposable sheath into a patient. A cystoscopic accessory may be inserted through a second inner lumen. The cystoscopic accessory traveling in a first direction from a proximal end of the sheath towards a distal end of the sheath and out of the distal end of the sheath. The cystoscopic accessory is redirected into a second direction as it leaves the distal end.

### BRIEF DESCRIPTION OF THE DRAWINGS

FIG. 1 is a front distal view of an embodiment of the present invention;

FIG. 2 is a cross-sectional side view of the embodiment shown in FIG. 1;

FIG. 3 is a cross-sectional side view of the embodiment in FIG. 1 in conjunction with a cystoscope;

FIG. 4 is a cross-sectional side view of the embodiment shown in FIG. 1 in conjunction with a cystoscopic accessory;

FIG. 5 is a front distal view of another embodiment of the present invention;

FIG. 6 is a cross-sectional side view of the embodiment shown in FIG. 5; and

FIG. 7 is a cross-sectional side view of an additional embodiment.

### DETAILED DESCRIPTION

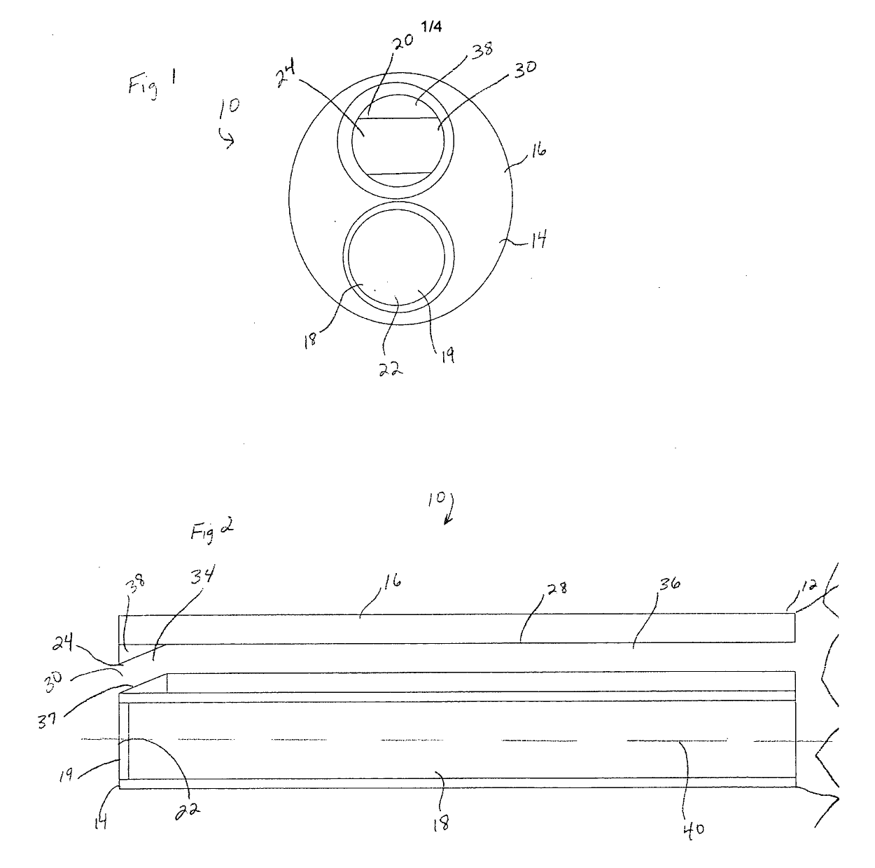

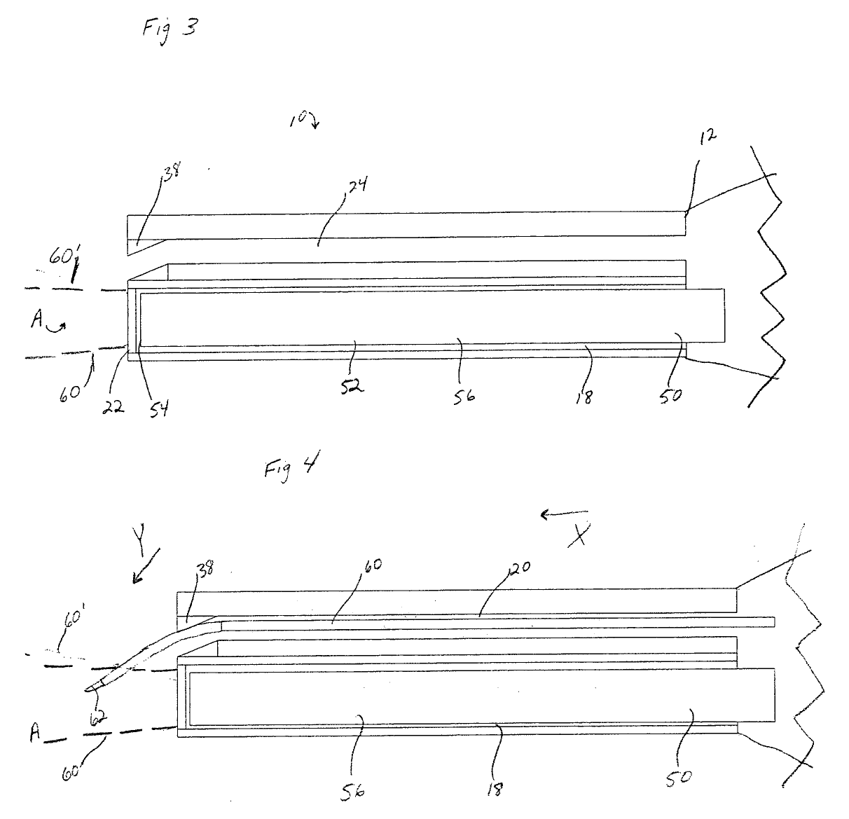

In one aspect of the present invention, as best seen in FIGS. 1 and 2, a protective disposable sheath 10 is provided to work in conjunction with a cystoscope or endoscope during therapeutic or diagnostic cystoscopic/endoscopic procedures. Sheath 10 is designed to work equally well with either a cystoscope or an endoscope and thus throughout this application, the two terms are used interchangeably unless specifically stated.

The sheath 10 includes a proximal end 12 and a distal end 14. The proximal/distal orientation is illustrated with regard to the proximity to which a particular end is positioned to a physician while the sheath 10 is being employed during surgery. Otherwise, the relative proximal end and distal end have no bearing with reference to a geometric spatial alignment. The sheath 10 is comprised of a flexible thin-walled structure 16 and includes an instrument receiving lumen 18 and a working lumen 20. The thin-walled structure 16 is preferably comprised of a polymeric material. The instrument receiving lumen 18 and the working lumen 20 may be integrally formed with the thin-walled structure 16 or the instrument receiving lumen and the working lumen may be separate elements disposed within the thin-walled structure.

The instrument receiving lumen 18 is adapted for use with an endoscope or cystoscope such that the particular scope employed may be disposed within the instrument receiving lumen with an end of the cystoscope being placed proximate the distal end 14 of sheath 10, as will be described below. The instrument receiving lumen 18 includes a first end 19 proximate the distal end 14 of sheath 10. A transparent wall 22 is disposed about the first end 19 of the instrument receiving tube 18 such that a window 23 is created at the first end of the lumen. The transparent wall 22 in conjunction with the flexible thin-walled structure prevents any debris or extraneous material from entering the instrument receiving lumen 18 thereby maintaining a sterile environment within the instrument receiving lumen. As a result of this separation, the instrument receiving lumen 18 is able to isolate at least a part of the cystoscope housed in the instrument receiving lumen from the external environment.

In the embodiment depicted in FIGS. 1 and 2, the first end of the instrument receiving lumen 18 has a generally circular cross-section. But the shape of cross-section of the instrument receiving lumen 18 may be altered and is primarily a function of the shape of the cross-section of the working end of the cystoscope employed, as will be described below.

In contrast to the instrument receiving lumen 18, the working lumen 20 includes an opening 24 disposed at the distal end 14 of the sheath 10. The opening 24 permits material to be passed from inside the working lumen 20 to outside the working lumen and the reverse. As with the instrument receiving lumen 18, the working lumen 20 is shown having a primarily circular cross-section although this is not required. The working lumen 20 is configured to permit various tools and instruments used during a cystoscopic surgery such as accessories required for a bioposy and/or fulguration, to be passed from the proximal end 12 of the sheath 10 through the length of the working lumen 20 and out opening 24. The working lumen 20 is comprised of a polymeric material and may differ from the material used to comprise the thin-walled structure 16.

In one aspect of the present invention, the working lumen 20 preferably includes a curved or slanted portion 34 that extends between a preferably relative straight portion 36 of the working lumen and opening 24. The slanted portion 34 may be constructed by placing an abutment 38 adjacent the interior wall 28 of the working lumen 20 such that the portion of the working lumen 20 proximate the distal end 14 of the sheath 10 forms the slanted portion 34. In addition, a portion of the interior wall 28 of the working lumen 20 closest to the instrument receiving lumen 18 may include a notch 37. The notch 37 may be similar in shape to the abutment 38. This enables the working lumen 20 to maintain its circular cross-section even through out/slanted portion 34. By placing the notch 37 near the instrument receiving lumen 18 and the abutment 38 opposite the instrument receiving lumen, the slanted portion 34 is angled towards the instrument receiving lumen and specifically towards the transparent wall 22. In one preferred embodiment, the slanted portion 34 is approximately 5-7 mm long with an angle of deflection relative to the straight portion 36 of the working lumen 20 being between approximately 10 and 30 degrees. Of course, the slanted portion may be longer or shorter and the angle of deflection may be less or more than 10 to 30 degrees.

The slanted portion 34 provides a passageway that diverts and directs any tools placed into the working lumen 20 from the proximal end 12 of the sheath 10 and through opening 24 in a direction towards a longitudinal axis 40 passing through the center of the instrument receiving lumen 18. Further illustration would be discussed below.

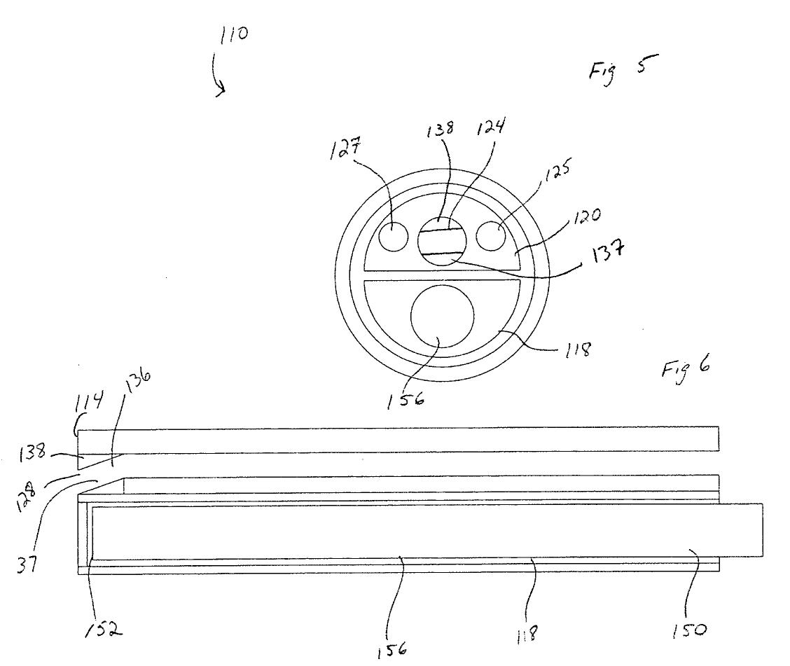

Referring to FIGS. 3 and 4, in a method of use, sheath 10 is disposed over a cystoscope 50 with a working end 52 of the cystoscope being disposed within the instrument receiving lumen 18 of the sheath. The working end 52 of the cystoscope 50 is preferably snug fit into the instrument receiving lumen 18 and a distal end 54 of the cystoscope is positioned proximate or even abuts the transparent wall 22 of the sheath 10. Although only a portion of the cystoscope 50 is shown, the cystoscope may be similar to the devices shown in U.S. Pat. Nos. 5,483,951; 6,174,280; and 5,447,148, the disclosures of which are hereby incorporated by reference herein. The endoscopes or cystoscopes shown in the U.S. patents mentioned are only illustrative of the types of instruments that may be used in conjunction with the sheath 10. The list should not be thought of as exhaustive, and various other endoscopes and cystoscopes may also be used with sheath 10.

In the embodiment shown in FIG. 3, the sheath 10 is shown working in conjunction with a cystoscope. The cystoscope 50 includes a conventional imaging device 56 disposed therein for conveying an image from the distal end 54 of the working end 52 of the cystoscope 50 to an eye piece (not shown in the figures) connected to a controlled body (not shown in the figures) of the cystoscope. As with most cystoscopes, since the imaging device 56 and specifically the lens of the imaging device is proximate and perpendicular to the transparent wall 22 of sheath 10, the line of sight through the imaging device 56 may be limited to the area A demarked by the dash lines 60 and 60′. Therefore, a physician looking through the eye piece of the cystoscope 50 may be limited in viewing only area A illustrated in FIG. 3, as transmitted by the imaging device 56. The physician may not be able to view the area outside of area A or only partially see an object outside of area A.

In a method of use, once the sheath 10 is placed over the cystoscope 50 and particularly the working end 52 of the cystoscope, the assembly may be introduced into the body. Upon reaching a desired location, the physician directs the imaging device towards a desired direction or object that they would like to view. Next, various tools that are required by a physician to perform a particular task such as a biopsy may be introduced through the proximal end 12 of the sheath 10 and specifically through the working lumen 20 of the sheath.

The tools, as for example, a cutting device 60, shown in FIG. 4, may be traversed in a first direction denoted by arrow X through the working lumen 20 until coming in contact with abutment 38. Preferably, the cutting instrument 60 or appropriate cystoscopic accessory is somewhat flexible. As the cutting instrument 60 is forced along the length of the working lumen 20 it comes into contact with abutment 38. As the cutting instrument 60 is still pushed in a direction from the proximal end 12 to the distal end 14 of the sheath 10 and is forced against the abutment 38, the abutment causes the cutting instrument 60 to deflect and slightly bend towards the instrument receiving lumen 18 of the sheath 10, and proceed in a second direction denoted by arrow Y, as shown in FIG. 4.

The actual size of the tools used for a cystoscopic surgery is much smaller than that shown in FIG. 4 but for illustration purposes are shown larger. Thus, although the deflecting of the cutting instrument 60 is illustrated as a relatively sharp angle so as to highlight this specific feature, in actuality the deflection of the cutting instrument may only include a relatively gentle bend or curve.

As a tip 62 of the cutting instrument 60 exits the working lumen 20 it is forced into a direction not only towards the instrument receiving lumen 18 but also preferably into a center of area A or at least within area A, which is being transmitted to an eyepiece or the like for viewing. Thus, while using the cutting instrument 60 to perform a biopsy or the like, the physician may clearly view the operation through the imaging device 56 and preferably in an approximate center of the eye piece.

By viewing the cutting instrument 60 or various other tools in a center of the eye piece, the sheath 10 along with the cystoscope 50 enable a physician to view and perform various operations while mimicking conventional cystoscope methods. This is because prior non-sheathed-cystoscopes generally placed the viewing lens proximate the working channel, such that the physician had a preferred observation location while performing intricate surgery. The sheath 10 once again provides a preferable viewing position for the physician while also isolating the cystoscope from coming into contact with any body fluids or the like during an operation, which would necessitate an expensive and time-consuming cleaning of the cystoscope.

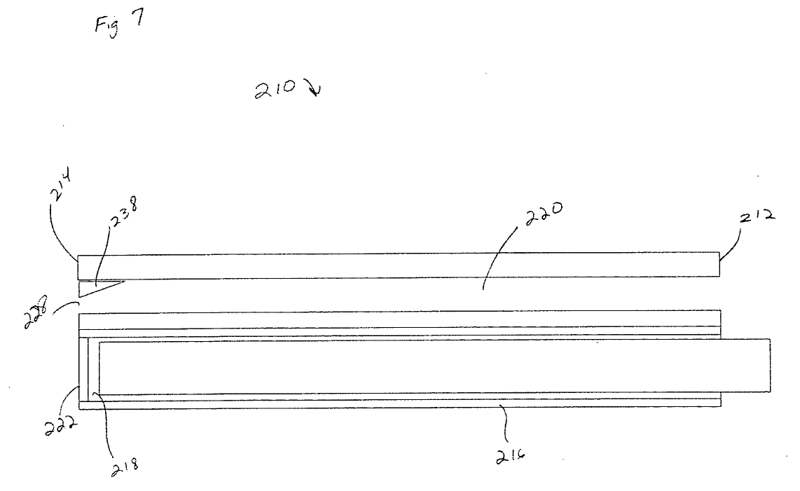

In an alternate embodiment of the present invention, the working lumen of the sheath may be provided with numerous channels for particular functions, as shown in FIGS. 5 and 6. In the embodiment shown in FIGS. 5 and 6, the working lumen 120 is divided into different channels; a working channel 124, an irrigation channel 125 and a biopsy channel 127.

The working channel 124 is configured to allow cystoscopic accessories such as cutting instruments and the like to be passed therethrough and out an opening 128 of the working lumen 120. The working channel 124 includes a slanted portion 134 created by notch 137 and abutment 138. As before, the slanted portion 134 is designed to steer and direct various tools passing through the working channel 124 into the direction of the instrument receiving lumen 118 and preferably into a position that may be captured by the imagining device, thereby allowing the physician to view the various instruments clearly while performing surgery.

The irrigation channel 125 provides a port and exit that allows liquids and the like to be passed into and out of the body on which surgery is being performed. And the biopsy channel 127 provides a pathway to remove a biopsy should the physician so require one.

Similar to sheath 10, sheath 110 includes an instrument receiving lumen 118 that is disposed over a working end 152 of a cystoscope 150. The cystoscope 150, as before, preferably includes an imaging sensing device 156 for enabling a physician to view a desired location.

Sheath 110 is substantially similar to sheath 10 and provides many of the same functions, except that the working tube 120 is divided onto individual channels for specific functions. In addition, the cross-section of both the instrument receiving lumen 118 and working lumen 120 are D-shaped. The D-shape components may be preferred depending on the specific cystoscope employed and specifically the shape of the working end 152 of the cystoscope 150. Of course, various alterations may be made to the sheath without deviating from the scope of the present invention.

In an alternate embodiment of the present invention, a sheath 210 may be similarly designed as previous embodiments, except that a notch may not be placed adjacent a working lumen 220 as shown in FIG. 7. Sheath 210, illustrated in FIG. 7 is similarly constructed to sheath 10, illustrated in FIGS. 1-4, and includes many of the same features including an instrument receiving lumen 218, a transparent wall 222 and a flexible thin-walled structure 216. One difference between sheath 210 and sheath 10 is that notch 37 included within sheath 10 is eliminated. Thus, as shown in FIG. 7, the working lumen 220 includes an abutment 238 that serves to redirect a cystoscopic accessory when pushed from distal end 212 of the sheath 210 to a distal end 214 of the sheath and out opening 228. The abutment 238 or angled portion is integrally formed with the working lumen 220. In this regard, all of the embodiments discussed herein may include a slanted portion, abutment or any other redirecting feature integral of formed separately from the inner lumen. Since the notch is not included with sheath 210, the dimensions of opening 228 of sheath 210 are smaller than the dimensions rest of the working lumen 220. As with the previous embodiment discussed here, sheath 210 is able to re-direct various instruments, such as a cutting instrument towards the instrument receiving lumen 218 by the use of abutment 238.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A disposable sheath to be used in conjunction with a cystoscope or endoscope, the disposable sheath comprising:
an external wall having a proximal end and a distal end;
a first channel disposed within said external wall, said first channel including an interior wall defining a hollow chamber and a longitudinal axis passing through a center of the first channel, said first channel having a redirecting portion at said distal end for redirecting a cystoscopic accessory in a desired direction as it moves through said redirecting portion.

2. The disposable sheath according to claim 1, wherein the cystoscope accessory is redirected toward a working end of the cystoscope that is disposed within the sheath.

3. The disposable sheath according to claim 1, wherein the redirecting portion includes a slanted wall.

4. The disposable sheath according to claim 3, wherein said redirecting portion includes an abutment defining said slanted wall and a notch opposite said abutment, said notch at least partially defined by an edge of said interior wall disposed within said slanted wall, both of which cooperate therewith to guide the cystoscope accessory in said desired direction.

5. The disposable sheath according to claim 1, further comprising a second channel having a second interior wall defining a second hollow chamber and a transparent wall proximate said distal end of said external wall, said second channel being adapted to fit over a working end of a cystoscope such that at least a portion of the working end of the cystoscope is isolated from an external environment.

6. The disposable sheath according to claim 1, wherein said first channel includes a first portion which has a circular cross-section.

7. The disposable sheath according to claim 1, wherein said first channel includes a first section that extends in a first plane and a second section that extends in a second plane, said second plane be angled relative to said first plane.

8. The disposable sheath according to claim 1, wherein said external wall is comprised of a polymeric material.

9. The disposable sheath according to claim 1, wherein said redirecting portion is integrally formed with said external wall.

10. A disposable sheath to be used in conjunction with a cystoscope or endoscope, the disposable sheath comprising:
a first inner lumen including a proximal end and a distal end, said first inner lumen further including a hollow chamber defined by an interior wall and an open end proximate said distal end, wherein an object may traverse from said proximal end of said hollow chamber toward said distal end in a first direction, said inner lumen including an abutment disposed adjacent said hollow member, said abutment having an edge, wherein when the object translating through said hollow chamber in a first direction contacts said edge of said abutment the object is redirected to a second direction; and
a second inner lumen including a proximal end, a distal end and a hollow chamber defined by an interior wall, said second inner lumen further having a closed transparent end positioned adjacent said distal end of said second inner lumen, wherein said second inner lumen is adapted to house a working end of a cystoscope such that the working end of the cystoscope is tightly surrounded and isolated from an external environment.

11. The disposable sheath according to claim 10, wherein said cross-section said hollow chambers of both said first inner lumen and said second inner lumen are substantially circular for at least a portion of each of said hollow chambers.

12. The disposable sheath according to claim 10, further comprising a cystoscope having a working end, said working end of said cystoscope including an imaging device for viewing an image, wherein when said working end of said cystoscope is disposed in said second inner lumen said imaging device imaging an area beyond said distal end of said second inner lumen such that one my view the area, wherein when the object is translating though said hollow chamber of said first inner lumen and redirected by said abutment to a second direction, said second direction steers said object towards the area being imaged by said imaging device.

13. The disposable sheath according to claim 12, wherein the object being translated through said hollow chamber of said first inner lumen is a cutting instrument.

14. The disposable sheath according to claim 10, further comprising at least one more inner lumen.

15. The disposable sheath according to claim 10, wherein the cross-section of the combination of said first inner lumen and said second inner lumen is substantially circular.

16. The disposable sheath according to claim 10, wherein said abutment is positioned proximate said distal end of said first inner lumen.

17. A method of performing a surgery, the method comprising the steps of:
placing a working end of a cystoscope into a first inner lumen of a disposable sheath;
inserting a portion of the cystoscope disposed within the disposable sheath into a patient;
inserting a cystoscopic accessory through a second inner lumen, the cystoscopic accessory traveling in a first direction from a proximal end of the sheath towards a distal end the sheath and out of the distal end of the sheath; and
redirecting the cystoscopic accessory into a second direction as it leaves said distal end.

18. The method of performing a surgery according to claim 17, wherein the working end of the cystoscope includes an imaging device that images a view of an area, wherein when the cystoscopic accessory is redirected to the second direction it moves toward the imaged area.

19. The method of performing a surgery according to claim 17, wherein the sheath includes at least one more inner lumen for use during the surgery.

20. The method of performing a surgery according to claim 17, wherein the second inner lumen includes a straight portion that is substantially parallel with the first inner lumen and a second portion that is angled relative to the straight portion.
